(19) United States
(12) Patent Application Publication
(10) Pub. No.: US 2011/0142758 A1
(43) Pub. Date: Jun. 16, 2011

(54) INDOLYL-PYRIDAZINYL-DIAZABICYCLONONANE DERIVATIVES IN LABELLED AND UNLABELLED FORM AND THEIR USE IN DIAGNOSTIC METHODS

(75) Inventors: Dan Peters, Malmo (SE); Daniel B. Timmermann, Herlev (DK); Lars Christian Rønn, Hillerod (DK); Elsebet Østergaard Nielsen, Kobenhavn K (DK)

(73) Assignee: NEUROSEARCH A/S, Ballerup (DK)

(21) Appl. No.: 12/995,625

(22) PCT Filed: Jun. 9, 2009
(86) PCT No.: PCT/EP09/57063

§ 371 Date: Jan. 14, 2011

(30) Foreign Application Priority Data
Jun. 10, 2008 (DK) PA 2008 00803

(51) Int. Cl. A61K 51/04; C07D 471/08

(52) U.S. Cl. 424/1.89; 544/238; 424/1.81; 424/1.85

(57) ABSTRACT

The present invention relates to indolyl-pyhdazinyl-diazabicyclononane derivatives in their labelled and unlabelled form. Furthermore, the present invention relates to the use of said derivatives in their labelled or unlabelled form in diagnostic methods, in particular for in vivo receptor imaging (neuroimaging).

TECHNICAL FIELD

The present invention relates to indolyl-pyridazinyl-diazabicyclononane derivatives in their labelled and unlabelled form. Furthermore, the present invention relates to the use of said derivatives in their labelled or unlabelled form in diagnostic methods, in particular for in vivo receptor imaging (neuroimaging).

### BACKGROUND ART

Neuroimaging is the use of certain technologies to measure a brain function or an aspect related to the functioning of certain parts of the brain, and enables the processing of information by centers in the brain to be visualized directly. Neuroimaging often requires the use of radioligands which have desirable properties for in vivo receptor imaging. These criteria include ease of labelling with positron-emitting radionucleotides, low rates of peripheral metabolism, high selectivity for brain regions holding the neuroreceptor of interest, and relatively high specific/non-specific binding ratios.

WO 2007/065892 and WO 2007/090888 describe certain pyridazinyl-diazabicyclononane derivatives, which are found to be cholinergic ligands at the nicotinic acetylcholine receptors and modulators of the monoamine receptors and transporters. However, the indolyl-pyridazinyl-diazabicyclononane derivatives of the present invention, in labelled or unlabelled form, are not reported.

### SUMMARY OF THE INVENTION

It is an object of the present invention to provide a compound that can be used to monitor in vivo and in vitro levels of nicotinic acetylcholine receptors, and in particular the nicotinic a7 receptor subtype, by way of non-invasive determination of the localisation of such receptors (neuroimaging).

This object is solved by providing an indolyl-pyridazinyl-diazabicyclononane derivative represented by Formula I

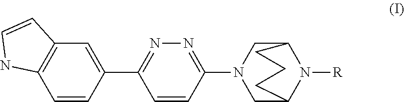

any of its enantiomers or any mixture of its enantiomers, or a pharmaceutically acceptable salt thereof, wherein

R represents hydrogen, or a labelled C₁₋₆-alkyl group.

In another aspect, the invention provides pharmaceutical compositions comprising a diagnostically effective amount of a labelled indolyl-pyridazinyl-diazabicyclononane derivative of the invention, or a pharmaceutically acceptable addition salt thereof, together with at least one pharmaceutically acceptable carrier or diluent.

In a further aspect the invention provides methods for the non-invasive determination of the distribution of a tracer compound inside a whole, intact living animal or human body using a physical detection method, wherein the tracer compound is a compound of the invention, or any of its enantiomers or any mixture thereof, or a pharmaceutically acceptable salt thereof, in labelled or unlabelled form.

Other objects of the invention will be apparent to the person skilled in the art from the following detailed description and examples.

### DETAILED DISCLOSURE OF THE INVENTION

### Labelled or non-labelled Indolyl-pyridazinyl-diazabicyclononane derivatives

In its first aspect the invention provides a novel indolyl-pyridazinyl-diazabicyclononane derivative represented by Formula I

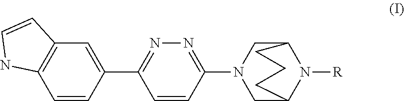

any of its enantiomers or any mixture of its enantiomers, or a pharmaceutically acceptable salt thereof, wherein R represents hydrogen, or a labelled C₁₋₆-alkyl group.

In a more preferred embodiment R represents hydrogen.

In a more preferred embodiment R represents a labelled C₁₋₆-alkyl group, and in particular a labelled methyl group.

In a most preferred embodiment the indolyl-pyridazinyl-diazabicyclononane derivative of the invention is

3-[6-(1H-Indol-5-yl)-pyridazin-3-yl]-3,9-diaza-bicyclo[3.3.1 ]nonane;

any of its enantiomers or any mixture of its enantiomers, or a pharmaceutically acceptable salt thereof.

In another most preferred embodiment the indolyl-pyridazinyl-diazabicyclononane derivative of the invention is

3-[6-(1H-Indol-5-yl)-pyridazin-3-yl]-[9-methyl-¹¹C]-3,9-diaza-bicyclo[3.3.1 ]nonane;

any of its enantiomers or any mixture of its enantiomers, or a pharmaceutically acceptable salt thereof.

### Labelled Compounds

In the context of this invention a labelled compound has one or more atoms replaced by an atom having an atomic mass or mass number different from the atomic mass or mass number usually found in nature. Such labelling will allow easy quantitative detection of the compound in question.

The labelled compounds of the invention may be useful as diagnostic tools, radio tracers, or monitoring agents in various diagnostic methods, and for in vivo receptor imaging.

The labelled isomer of the invention preferably contains at least one radionuclide as a label. Positron emitting radionuclides are all candidates for usage. In the context of this invention the radionuclide is preferably selected from ²H (deuterium), ³H (tritium), ¹¹C, ¹³C, ¹⁴C, ¹³¹I, ¹²⁵I, ¹²³I and ¹⁸F.

The physical method for detecting the labelled isomer of the present invention may be selected from Position Emission Tomography (PET), Single Photon Imaging Computed Tomography (SPECT), Magnetic Resonance Spectroscopy (MRS), Magnetic Resonance Imaging (MRI), and Computed Axial X-ray Tomography (CAT), or combinations thereof.

### Definition of Substituents

In the context of this invention an alkyl group designates a univalent saturated, straight or branched hydrocarbon chain. The hydrocarbon chain preferably contain of from one to eighteen carbon atoms (C₁₋₁₈-alkyl), more preferred of from one to six carbon atoms (C₁₋₆-alkyl; lower alkyl), including pentyl, isopentyl, neopentyl, hexyl and isohexyl. In a preferred embodiment alkyl represents a C₁₋₄-alkyl group, including butyl, isobutyl, secondary butyl, and tertiary butyl. In another preferred embodiment of this invention alkyl represents a C₁₋₃-alkyl group, which may in particular be methyl, ethyl, propyl or isopropyl.

### Steric Isomers

It will be appreciated by those skilled in the art that the compounds of the present invention may exist in different stereoisomeric forms, including enantiomers, diastereomers, as well as geometric isomers (cis-trans isomers). The invention includes all such stereoisomers and any mixtures thereof including racemic mixtures.

Racemic forms can be resolved into the optical antipodes by known methods and techniques. One way of separating the enantiomeric compounds (including enantiomeric intermediates) is—in the case the compound being a chiral acid—by use of an optically active amine, and liberating the diastereomeric, resolved salt by treatment with an acid. Another method for resolving racemates into the optical antipodes is based upon chromatography on an optical active matrix. Racemic compounds of the present invention can thus be resolved into their optical antipodes, e.g., by fractional crystallisation of D- or L- (tartrates, mandelates, or camphorsulphonate) salts for example.

Additional methods for the resolving the optical isomers are known in the art. Such methods include those described by Jaques J, Collet A, & Wilen S in “Enantiomers, Racemates, and Resolutions”, John Wiley and Sons, New York (1981).

Optical active compounds can also be prepared from optically active starting materials or intermediates.

### Pharmaceutically Acceptable Salts

The diazabicyclic aryl derivative of the invention may be provided in any form suitable for the intended administration. Suitable forms include pharmaceutically (i.e. physiologically) acceptable salts, and pre- or prodrug forms of the chemical compound of the invention.

Examples of pharmaceutically acceptable addition salts include, without limitation, the non-toxic inorganic and organic acid addition salts such as the hydrochloride derived from hydrochloric acid, the hydrobromide derived from hydrobromic acid, the nitrate derived from nitric acid, the perchlorate derived from perchloric acid, the phosphate derived from phosphoric acid, the sulphate derived from sulphuric acid, the formate derived from formic acid, the acetate derived from acetic acid, the aconate derived from aconitic acid, the ascorbate derived from ascorbic acid, the benzenesulphonate derived from benzensulphonic acid, the benzoate derived from benzoic acid, the cinnamate derived from cinnamic acid, the citrate derived from citric acid, the embonate derived from embonic acid, the enantate derived from enanthic acid, the fumarate derived from fumaric acid, the glutamate derived from glutamic acid, the glycolate derived from glycolic acid, the lactate derived from lactic acid, the maleate derived from maleic acid, the malonate derived from malonic acid, the mandelate derived from mandelic acid, the methanesulphonate derived from methane sulphonic acid, the naphthalene-2-sulphonate derived from naphtalene-2-sulphonic acid, the phthalate derived from phthalic acid, the salicylate derived from salicylic acid, the sorbate derived from sorbic acid, the stearate derived from stearic acid, the succinate derived from succinic acid, the tartrate derived from tartaric acid, the toluene-p-sulphonate derived from p-toluene sulphonic acid, and the like. Such salts may be formed by procedures well known and described in the art.

Other examples of pharmaceutically acceptable addition salts include, without limitation, the non-toxic inorganic and organic acid addition salts such as the hydrochloride, the hydrobromide, the nitrate, the perchlorate, the phosphate, the sulphate, the formate, the acetate, the aconate, the ascorbate, the benzenesulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate derived, the phthalate, the salicylate, the sorbate, the stearate, the succinate, the tartrate, the toluene-p-sulphonate, and the like. Such salts may be formed by procedures well known and described in the art.

Other acids such as oxalic acid, which may not be considered pharmaceutically acceptable, may be useful in the preparation of salts useful as intermediates in obtaining a chemical compound of the invention and its pharmaceutically acceptable acid addition salt.

Metal salts of a chemical compound of the invention include alkali metal salts, such as the sodium salt of a chemical compound of the invention containing a carboxy group.

In the context of this invention the “onium salts” of N-containing compounds are also contemplated as pharmaceutically acceptable salts. Preferred “onium salts” include the alkyl-onium salts, the cycloalkyl-onium salts, and the cycloalkylalkyl-onium salts.

Preparation of labelled indolyl-pyridazinyl-diazabicyclononane derivatives

The indolyl-pyridazinyl-diazabicyclononane derivatives of the invention may be prepared by conventional methods for chemical synthesis, e.g. those described in the working examples.

The end products of the reactions described herein may be isolated by conventional techniques, e.g. by extraction, crystallisation, distillation, chromatography, etc.

### Neuroimaging

The indolyl-pyridazinyl-diazabicyclononane derivatives of the invention are useful as diagnostic tools or monitoring agents in various diagnostic methods, and in particular for in vivo receptor imaging (neuroimaging).

In another aspect of the invention, a method for the non-invasive determination of the distribution of a tracer compound inside a whole, intact living animal or human body using a physical detection method is provided. According to this method a tracer compound is a compound of the invention, or any of its enantiomers or any mixture thereof, or a pharmaceutically acceptable salt thereof, in labelled or unlabelled form.

In a preferred embodiment the physical detection method is selected from Positron Emission Tomography (PET), Single Photon Emission Computed Tomography (SPECT), Magnetic Resonance Spectroscopy (MRS), Magnetic Resonance Imaging (MRI), Computed Axial Tomography (CAT), Computed Tomography (CT), Functional Magnetic Resonance Imaging (fMRI), or combinations thereof.

The labelled compound of the invention preferably contains at least one radionuclide as a label. Positron emitting radionuclides are all candidates for usage. In the context of this invention the radionuclide is preferably selected from ²H (deuterium), ³H (tritium), ¹¹C, ¹³C, ¹⁴C, ¹⁵O, ¹³N, ¹²³I, ¹²⁵I, ¹³¹I, ¹⁸F and ^{99m}Tc.

Examples of commercially available labelling agents, which can be used in the preparation of the labelled compounds of the present invention are [¹¹C]O₂, ¹⁸F, and NaI with different isotopes of Iodine. In particular [¹¹C]O₂ may be converted to a [¹¹C]-methylating agent, such as [¹¹C]H₃I or [¹¹C]-methyl triflate.

The tracer compound can be selected in accordance with the detection method chosen.

In one preferred embodiment, the compounds of the invention labelled by incorporation of a isotope into the molecule, which may in particular be an isotope of the naturally occurring atoms including ²H (deuterium), ³H (tritium), ¹¹C, ¹³C, ¹⁴C, ¹⁵O, ¹³N, ¹²³I, ¹²⁵I, ¹³¹I, ¹⁸F and ^{99m}Tc, and the isotope incorporation may be measured by conventional scintillation counting techniques.

In another preferred embodiment, the physical method for detecting said tracer compound of the present invention is selected from Positron Emission Tomography (PET), Single Photon Emission Computed Tomography (SPECT), Magnetic Resonance Spectroscopy (MRS), Magnetic Resonance Imaging (MRI), Computed Axial Tomography (CAT), Computed Tomography (CT), Functional Magnetic Resonance Imaging (fMRI), or combinations thereof.

In a more preferred embodiment the compound of the invention is labelled by incorporation of ¹¹C, ¹³C or ¹⁴C, and the isotope incorporation is measured by Positron Emission Tomography (PET) or Single Photon Emission Computed Tomography (SPECT).

In an even more preferred embodiment the compound of the invention is labelled by incorporation of ¹¹C, and the isotope incorporation is measured by Positron Emission Tomography (PET).

Before conducting the method of the present invention, a diagnostically effective amount of a labelled or unlabelled compound of the invention is administered to a living body. The diagnostically effective amount of the labelled or unlabelled compound of the invention to be administered before conducting the in-vivo method for the present invention is within a range of from 0.1 ng to 100 mg per kg body weight, preferably within a range of from 1 ng to 10 mg per kg body weight.

### EXAMPLES

The invention is further illustrated with reference to the following examples, which are not intended to be in any way limiting to the scope of the invention as claimed.

### Example 1

### Preparatory Example

All reactions involving air sensitive reagents or intermediates were performed under nitrogen and in anhydrous solvents. Magnesium sulphate was used as drying agent in the workup-procedures and solvents were evaporated under reduced pressure.

### Diethyl meso-2,6-dibromopimeloate (Intermediate Compound 1)

Pimelic acid (240 g, 1.5 mol) was placed into a two-necked round bottom flask (1000 ml) fitted with a reflux condenser and an argon inlet. The reflux condenser was connected with two consecutive flasks (500 and 1000 ml). The first flask (500 ml) was placed in to dry ice-isopropanol vessel and the second was half filled with water for HCl absorption. Thionyl chloride (368 g, 3.09 mol) was added in three portions (180, 100 and 88 g) and stirred at 40° C. until gas elution ceased. Finally temperature was raised to 100° C., the first flask with liquid SO₂ was disconnected. The flask was fitted with dropping funnel and gas outlet. During 3 hours the flask was continuously irradiated with 300 W UV lamp and bromine (490 g, 3.06 mol) was added drop-wise. The HBr formed was absorbed in two consecutive water filled flasks (2×1000 ml). When HBr elution ceased, the dropping funnel was filled with absolute ethanol (200 ml) and carefully added drop-wise. The chilled solution was washed with water, aqueous sodium acetate and sodium thiosulfate. The separated organic phase was dried over sodium sulfate, filtrated and distilled in multiple portions (about 40 ml each) by a Buchi oven in vacuo (0.5-1.0 mbar) at 150° C. collecting the fraction from the third flask. Yield: 487 g (87%).

### 20 Diethyl cis-1-Methylpiperidine-2,6-dicarboxylate (Intermediate Compound 2)

Diethyl meso-2,6-dibromoadipoate (1) (236 g, 0.631 mol) was placed into a two necked round bottom flask (2000 ml) fitted with a reflux condenser and a thermometer, and was dissolved in absolute THF (400 ml) under argon. A solution of methylamine (62 g, 2.0 mol) in absolute THF (400 ml) was added to the solution of compound 1. The flask was placed in cold water, to prevent it from warming. The reaction mixture was stirred for 18 hours under argon, the separated N-methylammonium bromide was removed by filtration and washed thoroughly with THF. The filtrate was concentrated on a rotary evaporator under reduced pressure and the residue (156 g) was distilled in four portions (about 39 g each) by a Buchi oven in vacuo (0.1-0.4 mbar) at 125° C. (average distillation time 1 hour) collecting the fraction from the third flask. Yield of compound 2 127.5 g (83%) as a light-yellowish oil.

### 3-Benzyl-9-methyl-3,9-diazabicyclo[3.3.1]nonane-2,4-dione (Intermediate Compound 3)

A solution of diethyl cis-1-methylpiperidin-2,6-carboxylate (127.5 g, 0.524 mol) and benzylamine (57.8 g, 0.540 mol) in xylene (150 ml) was refluxed in a round-bottomed flask (250 ml) for 44 hours. The latter was equipped with a vertical air condenser (15 cm) followed by a Liebig condenser, allowing removal of ethanol from the reaction mixture. The xylene was removed under reduced pressure through a Liebig condenser, the oil bath temperature was elevated to 205° C. and the mixture was heated under argon for 20 hours. The obtained product was distilled in four portions (about 45 g each) by a Buchi oven in vacuo (0.1 mbar) at 160° C. (average distillation time 1 hours) collecting the fraction from the third flask. The three combined 3^{rd} fractions (96 g) were dissolved by boiling in 50 ml of ethyl acetate and allowed to crystallize at room temperature for 3 days. The crystalline material was filtered off, washed with a small amount of ethyl acetate and dried in vacuo to afford 39.5 g of the product as a white crystalline solid. The filtrate was concentrated and the residue crystallized from ethyl acetate (30 ml) at 4° C. for 2 days to yield 6.2 g of the same product. Yield of compound 3 was 45.7 g (34%), mp. 117-118° C.

### 3-Benzyl-9-methyl-3,9-diazabicyclo[3.3.1]nonane (Intermediate Compound 4)

To a solution of compound 3 (45.7 g, 0.177 mol) in 1,4-dioxane (400 ml) placed into a three-necked round bottom flask (1000 ml), LiAlH₄ (9.0 g, 0.237 mol) was added in small portions and the mixture was refluxed under argon for 18 hours. The reaction mixture was cooled to 80° C. and a mixture of water (9 ml) and 1,4-dioxane (40 ml) was dropped carefully into reaction flask (caution: vigorous hydrogen evolution). A fine suspension was cooled to room temperature and treated with KOH solution (20 g in 50 ml of water). The organic phase was decanted and concentrated was concentrated under reduced pressure. The residue was distilled on Büchi oven in vacuo (0.1 mbar) at 130° C. The third collecting flask contained 3,9-diazabicyclo[3.3.1]nonane 4 (29.2 g, 72%) as a viscous colourless oil.

### 9-Methyl-3,9-diazabicyclo[3.3.1]nonane (Intermediate Compound 5)

To a solution of compound 4 (28.7 g, 0.125 mol) in absolute ethanol (100 ml) was added 10% Pd/C catalyst (6.3 g) under argon. The solution was hydrogenated with H₂ at 60 bar and 100° C. for 16 hours. The solution was filtered of on a Büchner funnel, the filtrate was concentrated under reduced pressure on a rotary evaporator and the residue distilled on Buchi oven in vacuo (0.1 mbar) at 100° C. to afford compound 5 (8.5 g, 49%) as a colourless gel.

In relation to the preparation of intermediate compounds 1-5, further reference is made to II Farmaco 55 (8), August 2000, Pages 553-562.

### Method A

### 3-[6-(1H-Indol-5-yl)-pyridazin-3-yl]-9-methyl-3,9-diaza-bicyclo[3.3.1]nonane fumaric acid salt (Compound A)

A mixture of 3-(6-iodo-pyridazin-3-yl)-9-methyl-9-aza-bicyclo[3.3.1]nonane (0.50 g, 1.45 mmol), indole-5-boronic acid (0.47 g, 2.91 mmol), potassium carbonate, 1,3-propandiol (0.60 g, 4.36 mmol) and water (15 ml) was bubbled with nitrogen for 15 min. Bis(triphenylphosphine)palladium(II)chloride (61.2 mg, 0.088 mmol) was added followed by reflux for 40 h. Aqueous sodium hydroxide (30 ml, 1 M) was added and the mixture was extracted twice with dichloromethane (2×30 ml). Chromatography on silica gel with dichloromethane, 10% methanol and 1% aqueous ammonia as solvent gave the title compound. Yield 290 mg (60%). The corresponding salt was obtained by addition of a diethyl ether and methanol mixture (9:1) saturated with fumaric acid. Yield 350 mg (90%). LC-ESI-HRMS of [M+H]+ shows 334.2028 Da. Calc. 334.20317 Da, dev. −1.1 ppm.

### 3-[6-(1H-Indol-5-yl)-pyridazin-3-yl]-3,9-diaza-bicyclo[3.3.1]nonane-9-carboxylic acid tert-butyl ester (Intermediate Compound)

Was prepared according to Method A from 3-(6-bromo-pyridazin-3-yl)-3,9-diaza-bicyclo[3.3.1]nonane-9-carboxylicacid tert-butyl ester.

### Method B

### 3-[6-(1H-Indol-5-yl)-pyridazin-3-yl]-3,9-diaza-bicyclo[3.3.1]nonane fumaric acid salt (Compound B)

A mixture of 3-[6-(1H-indol-5-yl)-pyridazin-3-yl]-3,9-diaza-bicyclo[3.3.1]-nonane-9-carboxylic acid tert-butyl ester (2.5 g, 5.96 mmol), TFA (6.79 g, 59.6 mmol) and dichloromethane (50 ml) was stirred for 3 days. Aqueous sodium hydroxide (50 ml, 4 M) was added and the mixture was evaporated to dryness. Chromatography on silica gel with dichloromethane, 10% methanol and 1% 30 aqueous ammonia as solvent gave the title compound. Yield 145 mg (8%). The corresponding salt was obtained by from 50 mg of free base by addition of a diethyl ether and methanol mixture (9:1) saturated with fumaric acid. Yield 39 mg (57%). LC-ESI-HRMS of [M+H]+ shows 320.188 Da. Calc. 320.18752 Da, dev. 1.5 ppm.

### Method C

### 3-(6-Iodo-pyridazin-3-yl)-9-methyl-9-aza-bicyclo[3.3.1]nonane free base (Intermediate Compound)

A mixture of 9-methyl-3,9-diazabicyclo[3.3.1]nonane (4.0 g, 28.5 mmol), 3,6-diiodopyridazine (9.5 g, 28.5 mmol), diisopropylethylamine (7.4 g, 57.0 mmol) and dioxane (50 ml) was stirred at 75° C. for 4 days. Aqueous sodium hydroxide (75 ml, 1 M) was added, dioxane was evaporated and the mixture was extracted twice with dichloromethane (2×75 ml). Chromatography on silica gel with dichloromethane, 10% methanol and 1% aqueous ammonia as solvent gave the title compound. Yield 4.61 g (47%). Mp 163-166° C.

### 3-(6-Bromo-pyridazin-3-yl)-3,9-diaza-bicyclo[3.3.1]nonane-9-carboxylicacid tert-butyl ester (Intermediate Compound)

Was prepared according to Method C from 3,9-diaza-bicyclo[3.3.1]nonane-9-carboxylic acid tert-butyl ester.

### Example 2

### Labelling Method

Labelling of the 3,9-diaza-bicyclo[3.3.1]nonane derivative of the invention may be accomplished in analogy with the method described by e.g. Jensen et al. [Jensen S B, Bender D, Smith D F, Scheel-Krüger J, Nielsen E Ø, Olsen G M, Peters D & Gjedde A: Synthesis of (±) 3-(6-nitro-2-quinolinyl)-[9-methyl-11C]-3,9-diazabicyclo-[4.2.1]-nonane; J. Label. Compd. Radiopharm. 2002 181-189].

### Preparation of [¹¹C] Methyl Iodide

[¹¹C] Carbon dioxide is prepared by the ¹⁴N(ρ,α)¹¹C nuclear reaction using a nitrogen gas target and 16 MeV protons produced by a GE Medical Systems PETtrace cyclotron.

[¹¹C] Carbon dioxide is purged from the target in a stream of nitrogen gas and trapped on 4 Å molecular sieves. On heating, the [¹¹C]O₂ is released and passed through a solution of LiAlH₄ in anhydrous tetrahydrofuran (THF; 300 μl). On completion of [¹¹C]O₂ transfer, the THF is evaporated and 1 ml hydroiodic acid is added. On heating at 160° C. the [¹¹C] methyl iodide formed is distilled in a stream of nitrogen gas to a reaction vial containing the labelling precursor.

### Synthesis and purification of [¹¹C] labelled 3-[6-(1H-Indol-5-yl)-pvridazin-3-yl]-9-methyl-3,9-diaza-bicyclo[3.3.1]nonane (Compound A)

3-[6-(1H-Indol-5-yl)-pyridazin-3-yl]-3,9-diaza-bicyclo[3.3.1]nonane (Compound B) in the form of a free amine (1 mg) is dissolved in anhydrous dimethyl sulphoxide (DMSO; 300 μl), and reacted with [¹¹C]-methyl iodide and heated for 5 min at 130° C. The resulting N-[¹¹C]-methyl labelled [¹¹]C-compound is subsequently purified by HPLC. Removal of the HPLC solvent may be achieved by heating the [¹¹C]-labelled 3-[6-(1H-Indol-5-yl)-pyridazin-3-yl]-9-methyl-3,9-diaza-bicyclo[3.3.1]nonane containing fraction under reduced pressure. The labelled product is then formulated in saline or water (10 ml) and passed over a 0.22 μm membrane filter into a sterile vial.

1. An indolyl-pyridazinyl-diazabicyclononane compound represented by Formula I 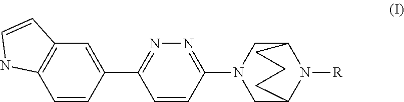
or a pharmaceutically acceptable salt thereof, wherein
R represents hydrogen, or a labelled C1-6-alkyl group.

2. The indolyl-pyridazinyl-diazabicyclononane compound of claim 1 which is
3-[6-(1H-Indol-5-yl)-pyridazin-3-yl]-3,9-diaza-bicyclo[3.3.1]nonane;
or a pharmaceutically acceptable salt thereof.

3. The indolyl-pyridazinyl-diazabicyclononane compound of claim 1 which is
3-[6-(1H-Indol-5-yl)-pyridazin-3-yl]-[9-methyl-11C]-3,9-diaza-bicyclo[3.3.1]nonane;
or a pharmaceutically acceptable salt thereof.

4. A pharmaceutical composition comprising a diagnostically effective amount of a labelled indolyl-pyridazinyl-diazabicyclononane compound of claim 1, or a pharmaceutically acceptable addition salt thereof, together with at least one pharmaceutically acceptable carrier or diluent.

5. A method for the non-invasive determination of the distribution of a tracer compound inside a whole, intact living animal or human body using a physical detection method, wherein the tracer compound is a compound of formula (I) according to claim 1, or any of its enantiomers or any mixture thereof, or a pharmaceutically acceptable salt thereof, in labelled or unlabelled form.

6. The method of claim 5, wherein the compound of the invention is labelled by incorporation of 11C, 13C or 14C, and the isotope incorporation is measured by Positron Emission Tomography (PET) or Single Photon Emission Computed Tomography (SPECT).
